(19) United States
(12) Patent Application Publication
(10) Pub. No.: US 2020/0282228 A1
(43) Pub. Date: Sep. 10, 2020

(54) SYSTEM AND METHODS FOR MAGNETO-MECHANICALLY RADIATING OSCILLATING MAGNETIC FIELDS FOR THERAPY AND SCIENTIFIC USES

(71) Applicant: Paul M. BODE, Harrison, AR (US)

(72) Inventor: Paul M. BODE, Harrison, AR (US)

(21) Appl. No.: 16/810,467

(22) Filed: Mar. 5, 2020

Related U.S. Application Data
(60) Provisional application No. 62/814,382, filed on Mar. 6, 2019.

(51) Int. Cl. A61N 2/12; H01F 7/02; H01F 7/20; H01F 7/06; A61N 2/00; A01G 7/04

(52) U.S. Cl. CPC A61N 2/12; H01F 7/02; A01G 7/04; H01F 7/064; A61N 2/006; H01F 7/20

(57) ABSTRACT

A device for magneto-mechanically radiating oscillating magnetic fields by periodically and precisely moving permanent magnets such that the field they continually radiate oscillates precisely. The device provides for varying magnetic field therapy as well as for use in other areas such as magnetic therapy for mammals and for improving plant growth. The device provides a varying magnetic field therapy that allows a user to use it without any change to the aesthetics of the location it is used in because the field can radiate through, or from under, objects.

### CROSS-REFERENCE TO RELATED APPLICATIONS

This application claims the benefit of and priority to U.S. Provisional Application No. 62/814,382 entitled “Serenity Sphere” filed on Mar. 6, 2019 and U.S. patent application Ser. No. 15/833,268 entitled “SYSTEM AND METHODS FOR DESIGN, OPERATION AND USE OF THE MAGNETIC RESONANCE OF THE EARTH FOR SLEEP ENHANCEMENT” filed on Dec. 6, 2017. The foregoing applications are incorporated herein by reference in their entirety.

### FIELD OF THE INVENTION

The invention is directed to systems and methods for generating sinusoidal oscillating magnetic fields in an area, typically the size of a small room, for use in magnetic therapy in humans and other mammals such as dogs and horses, and for scientific research with, but not limited to, botany.

### BACKGROUND OF THE INVENTION

Magnetic therapy is generally acknowledged as effective in many applications. For millennia people have been placing permanent magnets on bodies for therapeutic purposes. A popular story attributes Cleopatra's beauty to her use of lodestones placed on her face as she rested. In modern times, pulsed magnetics have been employed. The human body has been scientifically demonstrated as being sensitive to, and responsive to magnetic fields. An early example of the connection between the earth's magnetic field and human rhythms and health was described by Professor Rütger Wever. From 1964 to 1989 he conducted 418 experiments on 447 volunteers, mostly college students. In a bunker where he removed the influence of the earth's fields, he observed that a frequency of 7.8 hertz played a role in human physiological and psychological health. He removed this frequency from, and then reintroduced it back to, the students' environment. The student volunteers were in specially constructed shielded bunkers. Professor Wever demonstrated that this frequency affected the rhythms of the students, and when removed it had negative physiological and psychological effects.

Recent developments use pulsed electromagnetic field therapy (PEMFT), also known as low field magnetic stimulation (LFMS), for therapeutic purposes. In PEMFT, electromagnetic fields are used in an attempt to heal non-union fractures and depression. However, PEMFT suffers from disadvantages and this technique is not used for physiological and psychological effects of patients.

Additional techniques include magnetic therapy to alleviate pain and other health concerns.

A search of prior art reveals only one instance where permanent magnets are used to develop an oscillating magnetic wave. For completeness, coil-based systems were also researched. Known references are provided below:

U.S. Patents

| | Kind | | | |
|---|---|---|---|---|
| Patent Number | Code | Issue Date | Patentee | Title |
| 10,500,408 | N/A | Dec. 10, 2019 | The Methodist | Method and apparatus for |
| | | | Hospital, | providing transcranial magnetic |
| | | | Houston, TX | stimulation (TMS) to a patient |
| | | | Cornell | |
| | | | University | |
| | | | Ithaca, NY | |
| 9,649,502 | B2 | May 16, 2017 | James Phillips | Devices and methods of low |
| | | | Yi Jin | frequency magnetic stimulation |
| | | | | therapy. |
| 6,203,486 | B1 | Mar. 20, 2001 | Wendell Miller | Earth magnetic-field |
| | | | Robert South | augmenters |
| | | | Michael South | |
| 7,819,794 | B2 | Oct. 26, 2010 | Paul Becker | Method and apparatus for the |
| | | | | treatment of physical and |
| | | | | mental disorders with low |
| | | | | frequency, low flux density |
| | | | | magnetic fields |

U.S. Pat. No. 10,500,408 is the only reference found that employs an oscillating magnet. The magnets are attached to motors on a skull cap. The motors spin at a minimum of 24,000 RPM to create a 400 hz signal and is limited to trans-cranial applications. By employing frequencies >400 Hz in the context of a close (<1 inch) physiological environment, the approach essentially scrambles or blocks local natural human electro-physiology in that tissue. The patent exposes the tissue to gauss approximately 5000 times the guidelines for the public, and 1000 times for occupational exposure. These are in direct opposition to the approach herein. This invention uses frequencies <=17.2 hz, low gauss, and promotes natural process, over the entire body.

Of note, U.S. Pat. No. 10,500,408 addresses safety but does not address beat frequencies and phase differences that result from differences in the motor speeds which are inevitable. The beat frequencies can introduce frequencies not accounted for in the patent, a safety concern. The phase differences between the various motors may also cumulatively provide peaks of gauss not addressed in the patent. These concerns are by design, not applicable to the approach taken herein.

U.S. Pat. No. 9,649,502 requires a device to be placed on a head of a user and would be uncomfortable to sleep with.

U.S. Pat. No. 6,203,486 claims to augment the Earth's magnetic field but does so in a static manner, exceeding the strength of what naturally occurs.

U.S. Pat. No. 7,819,794 requires an object to be worn or a thick coil module to be placed near you, both intrusive in nature.

It is an object of the invention to address the deficiencies in the prior art.

### SUMMARY OF THE INVENTION

It is an object of the invention to radiate a varying magnetic field using permanent magnets as the source of the radiated magnetic field.

It is an object of the invention to precisely control the time domain of the magnet's position to radiate a purely magnetic wave of specific frequency.

It is an object of the invention to be able to control the strength of the radiated magnetic field through the combination of selected magnetic strength (Gauss), and control of distance from the unit.

It is an objective of this invention to provide oscillating magnetic fields to a target area or object without needing to encompass such areas or objects, or place near such, electric coils providing said magnetic field.

When used therapeutically, it is an object of this invention to provide the entire body with a uniformly oscillating magnetic field whose average strength is on the order of magnitude of the earth's own magnetic field.

When used therapeutically, it is an object of this invention to emulate frequencies, or fractions of frequencies that the earth itself oscillates at.

When used therapeutically, it is an object of this invention to stay within recognized guidelines for public exposure to magnetic fields.

Other objects of the invention are achieved by using a permanent magnet's flux, positioned over time by any one or more of mechanical, electrical or electronic means. The periodic positioning results in the magnet's field oscillating as felt by the intended target. The frequency of this oscillation is controlled to enhance natural processes within the body. As the whole body is naturally exposed to the earth's own magnetic oscillations, an objective of the invention is to replicate a magnetic field throughout a body of a person, rather than just focusing on the person's head. By exposing all tissues simultaneously to the one wave, the effect of promoting the wave in the body is enhanced.

In certain objects, the invention provides an area (for example: a bed, small room, kennel, stall), not just a cranium, with a single magnetic wave, not a myriad of waves. It is the single wave, not coordination between multiple waves, that is employed to achieve the advantages of embodiments of the invention.

As there is only a single source of the magnetic wave, no beat frequencies are introduced. The frequency of the magnet(s) corresponds to natural phenomena. The design and operation of are such that gauss limits are on the order of the earth's magnetic field and the invention is used such that guidelines for public exposure are adhered to.

It is another object of the invention is to allow the cells of a human body, animal or plant to experience the exact same magnetic wave in a time domain, meaning the induced stimulus in the target object occurs simultaneously throughout the body.

It is another object of the invention to provide a method of selecting appropriately sized permanent magnets and positioning them periodically in the time domain, to establish desired precise behavior in the frequency domain. The invention presents a physically large field, in an unobtrusive manner, that fluctuates in a manner to how the earth may influence the same area with its own magnetic field.

These and other objects of the invention are achieved by providing a varying magnetic field generating system comprising: a permanent magnet array comprised of one or more magnets, a positioning mechanism for the magnets, a control system for the positioning mechanism to achieve precise control over the positioning of the magnets in the time domain.

In certain embodiments, there may be a single magnet in the array.

In certain embodiments, the magnets are positioned using mechanical methods only, with no use of electrical energy.

In certain embodiments, the magnets are positioned using electrical motors.

In certain embodiments, the magnets are positioned by electrical coils.

In certain embodiments, there may be one, or more, coils used to position the magnet array.

In certain embodiments the frequency projected by the magnets is held at constant frequency.

In certain embodiments the frequency projected by the magnets is varied to suit scientific or therapeutic objectives.

In certain embodiments, the system is devoid of any smart phone, tablet or personal computer.

In certain embodiments, the system employs remote control technology such as Blue Tooth, LAN or WIFI.

In certain embodiments weaker or stronger magnet arrays may be used such as if developing a therapy recliner that uses magnets, not coils, or if applying the field to a dog pen or horse stall.

Other objects of the invention are achieved by providing a system for radiating an oscillating magnetic field, comprising: a magnet array consisting of one or more magnets, the one or more magnetic providing a radiated magnetic field through spinning or other periodic motion of the one or more magnets; a positioning mechanism for positioning the magnet array within the system; and a control system used to control the radiated magnetic field, wherein the system achieves precise control over the radiated magnetic field in a time domain.

Other objects of the invention are achieved by providing a method of providing a time-varying magnetic field to a subject in need thereof, the method comprising: providing a magnet array consisting of one or more magnets, a positioning mechanism, and a control system; using the control system to control the strength of the radiated magnetic field, such that the magnetic array is controlled by the rate of spinning or other periodic motion of the one or more magnets; and varying the strength of the radiated magnetic field in a time domain.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The figures provided here show the progression of embodiments from conception and proof of concept, to commercial production.

FIG. 1 shows the very first inception of the concept. The motor's current was controlled to limit the speed of the magnets through a geared transmission, thereby controlling the frequency of the radiated field.

FIG. 2 shows the first electronically controlled magnet array. The coil produces a weak magnetic field sufficient to turn the magnet over. A magnetic screw is used to tilt the rotor to ensure the magnet array does not come to rest at a zero-torque point.

FIG. 3 employs two coils to ensure starting torque, removing the need for a magnetic offset screw.

FIG. 4 shows the rotation shifted to a vertical axle approach.

FIG. 5 illustrates a typical magnetic control field used to drive the magnet array.

FIG. 6 shows the radiated magnetic flux at a distance of 2 feet in relation to the flux developed in the coils.

FIG. 7 provides empirical data showing the relationship between distance and magnet flux.

FIG. 8 compares a pulsed approach to a sinusoidal approach for radiating a magnetic field in terms of the rate of change of flux.

FIG. 9 compares the harmonic results and effects of the pulse and the sinusoidal approach.

FIG. 10 compares ICNIRP Guidelines for exposure to Magnetic Flux to distances from the face of the magnets in the current embodiment.

### DETAILED DESCRIPTION OF THE INVENTION

In the following description, numerous details are set forth for the purpose of explanation. However, one of ordinary skill in the art will realize that the invention may be practiced without the use of these specific details.

The invention, also called the “Sphere”, includes a mechanism to create a periodic motion coupled to a permanent magnet, or magnet array, such that the motion causes the magnetic field of the magnet(s) to experience the same periodic motion. The motion of the radiated magnetic field from the magnet is employed with an understanding of Faraday's laws of induction and Maxwell's field equations. Purely magnetic waves, of precise frequencies, with minimal to no harmonics can be radiated and employed.

From Faraday (circa 1830), we learned that an induced electromotive force from a time-varying magnetic field is proportional to the rate of change of the flux per unit time. Faraday employed coils in his lab and observed the phenomenon, coil to coil. A stationary permanent magnet does not produce an induced effect because there is no time-varying component to the field. To date, it was counter-intuitive to use a permanent magnet with a constant magnetic flux to produce a varying magnetic field.

Maxwell (circa 1860), a superior mathematician, then went on to define the surface integrals to show the relationship between time-varying magnetic fields in three dimensions, and the induced electromotive force.

Studies have measured the DC (zero Hz) resistivity (ohm-cm) of human tissue, generally establishing the conductivity of blood, heart and muscle tissue having higher conductivity than skeletal or fat tissue. Conductive materials when placed in varying magnetic fields produce voltages.

The permittivity of human tissue has also been studied and since the permittivity of human tissue is greater than zero, its frequency response is also not zero. The higher the frequency applied to tissues, generally, the lower the impedance of the tissue becomes.

Understanding the physics of magnetic induction, resistivity and permittivity indicates that external magnetic fields do induce voltage in tissue which are dependent on the frequency and magnitude of the magnetic field. The frequency component has a compounded effect because it influences both the magnitude of induction (proportional to the rate of change of magnetic flux per unit time) and the impedance of the tissue (current increasing with frequency as impedance decreases). The magnitude and frequency of these voltages affects physiology and neurology.

Dr. Wever demonstrated that what earth naturally produces works to our benefit, consistent with many observations in other areas of health. The principal way for this to happen is for the human body to develop electrical waves as a result of the influence of the earth's magnetic field. This invention develops oscillating magnetic wave fields that immerse the entire body, and uses frequencies associated with deep sleep, REM sleep or alert cognitive activity. The chosen frequencies coincidentally are frequencies the earth itself oscillates at various times, or lower fractions thereof. The body senses these just as it does natural magnetic waves, the physics of induction being indistinguishable between the two. These influence the natural waves in our body and promote the electrical waves we see during deep sleep, REM sleep or engaged cognitive activity. Care is taken to not introduce any un-natural high frequencies, particularly those associated with pulsed technology.

The invention, as stated earlier, is counter intuitive in that it uses permanent magnets to apply the time-varying magnetic field, to the whole body. In the context of magnetic therapy, there are distinct advantages to this. The inertia of the magnet and the use of non-ferrous or minimally ferrous cores for the coils in the current embodiment essentially eliminates any harmonics in the radiated magnetic field, producing a very natural wave. The wave can be radiated to the intended physical points without intruding upon the natural state of the location. So, for example, a person with this under their bed, or in an adjacent room against the wall next to their bed, has no intrusion on their sleeping space. The same principle can be applied to other scientific research applications.

Because the invention uses a powerful permanent magnet, much less energy is used. No energy is expended by the user to create and maintain the magnetic flux, only to move the magnet. Once the magnet is moving, the only energy required is to overcome friction and air resistance. The therapeutic effect ceases when the magnet stops moving. As an example, a room can be filled with an oscillating magnetic field the magnitude of the fluctuation being on the order of the earth's magnetic field, using a 5-volt USB power supply or USB battery. As in nature, a person's entire body is simultaneously experiencing this wave. Similarly, it can be placed under a chair, bed, couch, or on a desk or conference table, and have multiple people experience the same magnetic wave.

Because the radiated magnetic wave is produced by a physical object moving, not through a coil driven by electronic circuitry, a more natural effect is achieved with minimal to no harmonic effects associated with rapid pulse edges occurring in many magnetic therapy devices.

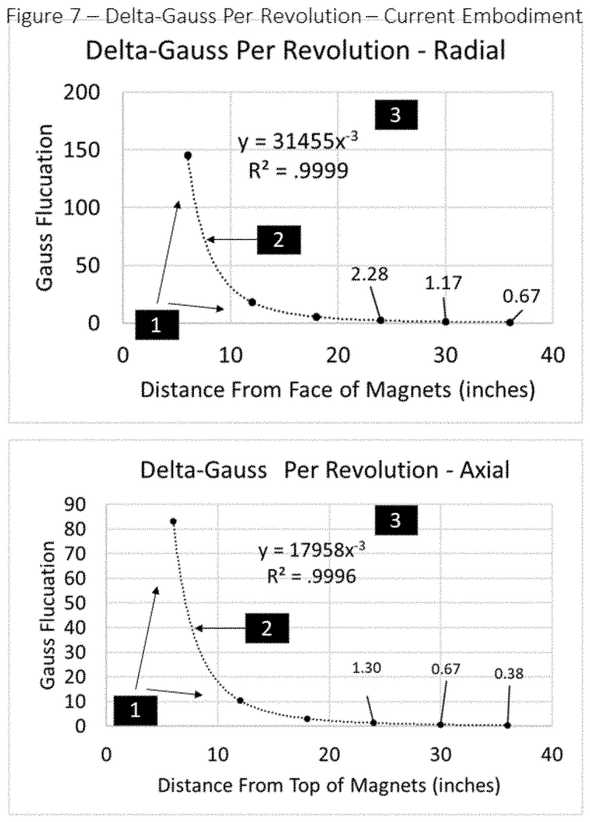

FIG. 7 shows the Gauss fluctuation as a function of distance from the invention as currently embodied. The absolute magnitude of flux is essentially half of the delta-Gauss per revolution. The current embodiment is designed for under-bed or in-office use, at a nominal distance of 3 feet and suggested use between 2 and 5 feet.

A further embodiment is provided where weaker magnet arrays are used and placed within a recliner, making the optimal distance match the geometry of a person sitting in the chair.

A further embodiment is provided for stronger magnets, covering larger areas.

At 3 feet, the Gauss strength from the magnet is approximately 0.337 Gauss (Earth itself is around 0.500 Gauss), and the fluctuation is approximately twice that at 0.675, Gauss, peak to peak. The inductive capability of a purely magnetic wave is proportional to its amplitude (the static strength of the Gauss filed at a given distance), and the maximum slope of the associated rate of change of the magnetic field. Herein lies an understanding of why this approach is superior, safer and more natural than most pulsed systems. In pulsed systems, the rate of change of the flux is very steep, and huge fluxes are applied. This creates relatively huge spikes in the target object. Yet the temporal aspect is very short where the period of time of the rate of change of flux is very short. Work performed by the field is a product of both the time of, and the magnitude of the pulse (analogous to an understanding of impulse equations force x time). In pulsed systems a huge gauss strength is needed to offset the very short period of time of the flux changing (often measured in uSec). In the approach this invention uses, the equivalent RMS effect can be achieved with much lower Gauss because the flux is changing constantly, sinusoidally (naturally). The changing flux is a result of a physically moving or spinning object, a very natural phenomenon, much like the Earth itself. The Earth's geo-electric waves propagating around the globe also impact the geomagnetic fields. The geo-resonance achieved in the atmosphere affects the magnetosphere.

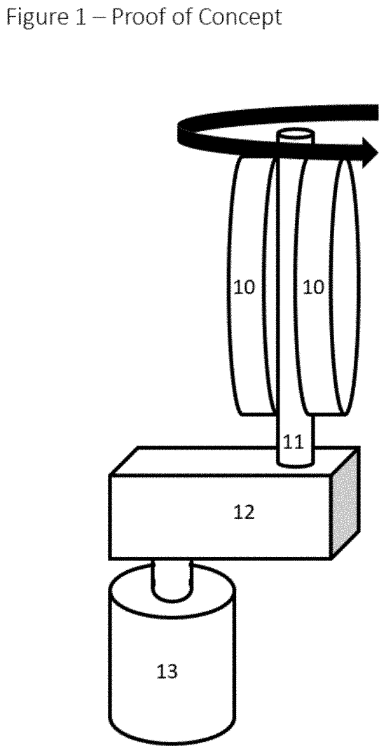

FIG. 1 shows a first embodiment. It shows a small DC motor, coupled through a mechanical geared transmission to reduce the revolutions per minute (rpm). The output shaft has a round permanent magnet attached to it such that the rpm of the magnet is proportional to the rpm of the small motor of which the current can be controlled to control rpm. This was initially put aside for development because the geared transmission was too noisy for use in a bedroom. Effort was made to develop Serenity Wave Halo instead, as indicated in the previous patent claims credited here in.

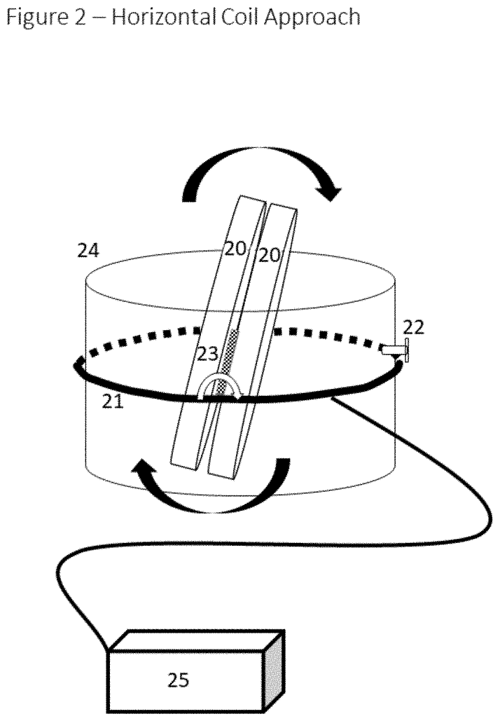

FIG. 2 shows another embodiment of the invention. It consists of an array of permanent magnets which can rotate around a horizontal axis. A single coil is used to drive the rotation of the magnet. The associated software accounts for the inertia of the magnet and times the polarity in the coil to provide a constant unidirectional torque. The significant advantage of simplicity was offset by the randomness of starting, and the occasional non-starting if the magnet came to rest perfectly horizontal. A magnetic screw was applied to never allow the magnet to come to rest horizontally which increased the starting reliability, but this introduced vibrations as the magnet spun past the screw.

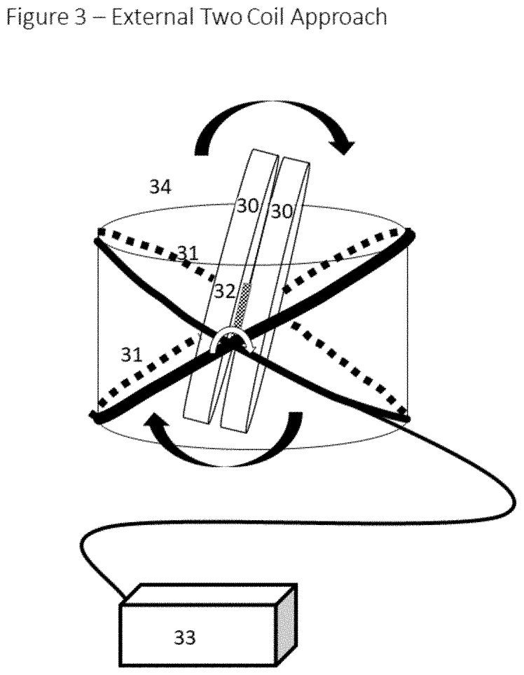

FIG. 3 shows the next embodiment where an additional coil was added to ensure a constant starting torque could always be applied. This increased the complexity of design but ensured a consistent start and rotational direction. With a consistently starting unit, focus was then applied to precise control of the rotation.

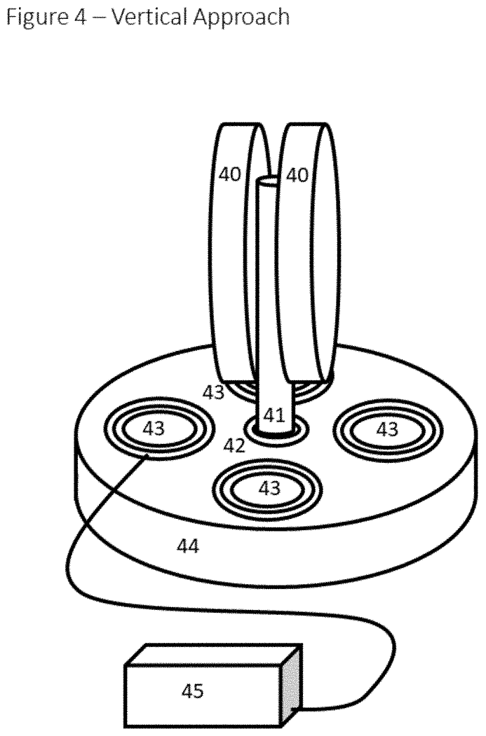

FIG. 4 shows the next embodiment where the axle was shifted vertically, and a nylon bearing system was developed to ensure a silent movement at theta band and delta band sleep frequencies. The vertical axle approach made it easier to address any imperfections of the center of mass of the magnets and reduced axle friction. This embodiment employed systems using two, three and four coils, and concluded that four coils, completing two electrical circuits 90 degrees apart, with alternating polarity, provided four points of torque per revolution. The coils are beneath the magnet, producing a weak magnet field powered off a USB power source. The proximity of the magnet to this weak field is sufficient to allow for magnetic lock between the two. The magnet then amplifies the time-based component of the weak magnetic field projecting it out for use.

As currently applied, this invention is helping people sleep better and focus better by stimulating natural waves within the body. The control unit applies select naturally occurring geo-magnetic frequencies, or fractions thereof, to produce a delta-band wave, a theta-band wave and a beta-band wave. The success of this device is a result of the fact the body experiences a gentle purely magnetic wave, of a constant frequency, of sufficient duration to influence the electric waves within the body.

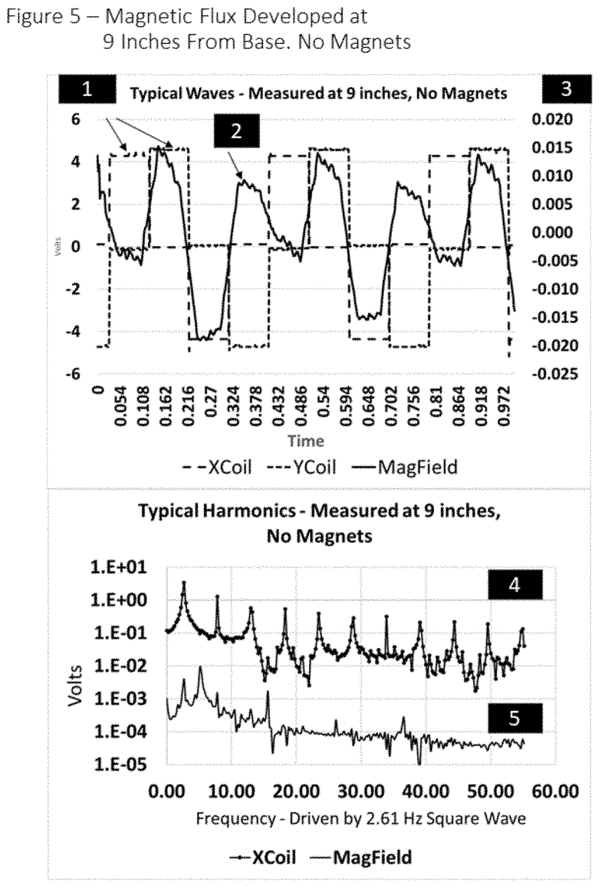

FIGS. 5, 6 and 7 represent data collected from running Spheres using various instruments including a calibrated Alpha Lab Vector/Magnitude Gaussmeter Model VGM, the magnetic sensor on a TriField Natural EM meter, also from Alpha Lab, a calibrated Tektronix Digital Oscilloscope Model TBS-1052B and a Rigol Digital Oscilloscope model 1054Z. The Alpha Labs meter is used to quantify gauss levels. The other instruments are used for harmonic analysis only, exporting data directly to Excel, or through the oscilloscope, to Excel. Their values are for illustration only. All values may vary by embodiment.

FIG. 5 illustrates a typical magnetic control field used to drive the magnet array. It shows the square wave voltage and the flux developed in the top graph. The bottom graph shows the expected harmonics from this pulsed approach. These graphs specifically reference a 2.61 Hz frequency, ⅓ of the well-known 7.83 Hz earth resonance. Of note, these harmonics are not transmitted by the magnet array, a distinct advantage of this approach, as other systems expose the target to the harmonics. This is evident in the next figure. The voltages may vary by embodiment and are dependent on the voltage applied to the coil and the voltage developed by the magnetic sensor system used. The harmonic behavior is common across embodiments.

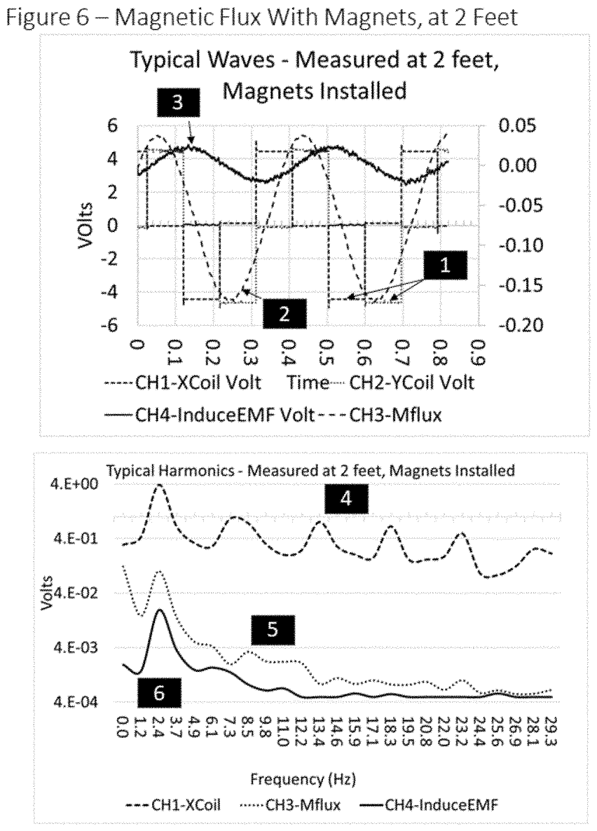

FIG. 6 shows the radiated magnetic flux at 2 feet in relation to the flux developed in the coils. The square wave harmonics used to drive the magnet array are eliminated by the physical inertia of the spinning magnet much like a flywheel smooths out each piston's contribution in a combustion engine. The top graph shows the [1] voltage/flux square wave applied to/developed by the coil and shows the [2] sinusoidal wave form of both the magnetic field, and [3] induced voltage in a sensing coil, at 2 feet. A simple air core copper coil 6 inches in diameter is used to characterize the nature of the magnet array's ability to induce a voltage.

The bottom graph shows the [4] harmonics of one of the two drive coils (both being identical in nature, offset 90 degrees), [5] the harmonics of the magnetic field, and the [6] harmonics of the induced voltage. Note the harmonics evident in curve [4] are essentially removed in curve [6]. The weak magnetic flux of the drive coils is amplified and physically filtered by the magnet array.

FIG. 7 shows how the strength of the magnet array's field diminishes as a function of distance from the array. Datapoints [1] are radially (top chart) and axially (bottom chart) out from the magnets as the array turns on its axis. Curve [2] is the trend curve to fit the data. It shows the data collected matches the type of decay we expect to see as a function of distance. The coefficient of determination (R\hat{ }2) [3] is for curve [2]. The high R\hat{ }2 values show the expected nature of the function being accurately measured.

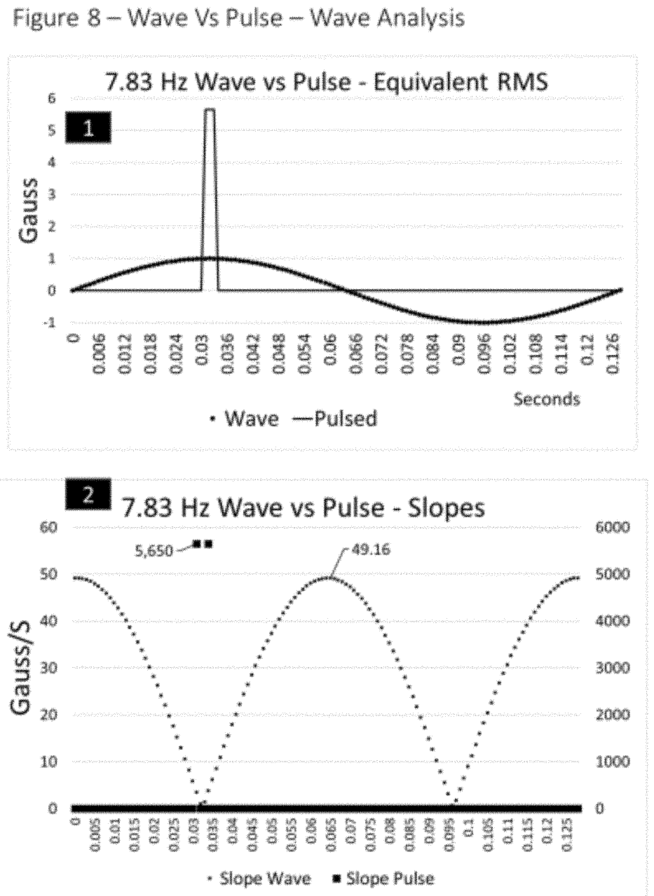

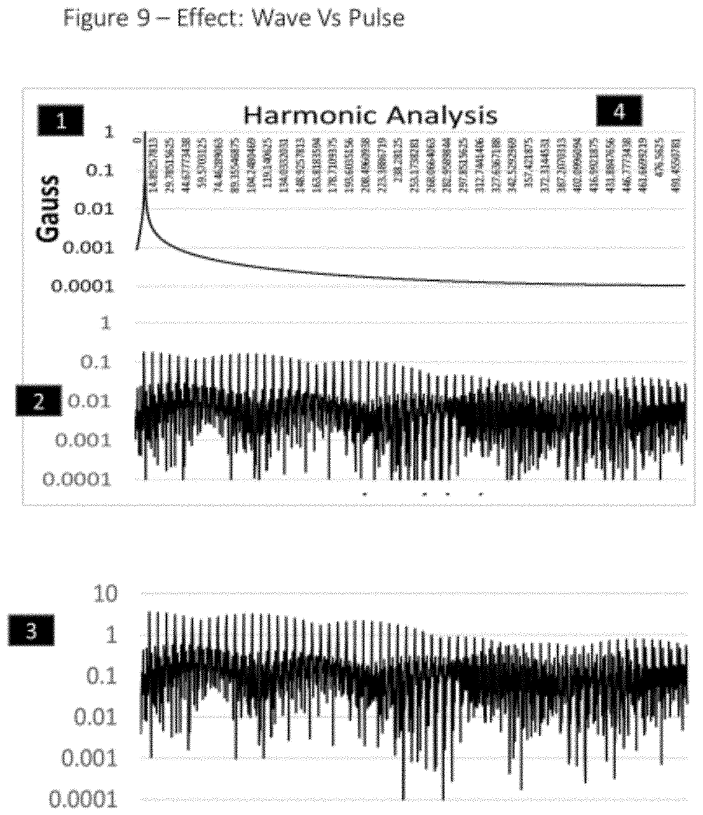

FIGS. 8 and 9 form the basis for subsequent discussion of effectiveness of the invention and advantages.

In FIG. 8, charts [1] and [2] represent a pulsed and a sinusoidal approach to creating a magnetic (Gauss) wave with a 0.127 second period. Chart [1] shows the magnitude of the gauss applied, pulse and sine wave. Their magnitudes were set to have equivalent RMS values. Chart [2] shows the rate of change of flux using 1 mS increments of time to illustrate a point. The magnetic field's ability to influence an object or area is proportional to the rate of change of the flux (from Faraday's law). The pulsed curve's flux only has an impact while it is rising, and when it is falling. There are only 2 discreet points of influence related to induced EMF. While the pulse is at its maximum, it does not induce voltage. The sine wave is continually changing over the period. There are 126 points of influence, ignoring the two zero points. In the context of physiological effects, the duration of the transient has a compounded effect on effectiveness. The same intensity applied twice as long will have more than twice the effect. So, while the pulse shows an instantaneous rate of change of flux 2 orders of magnitude higher, because of the very short period of time it actually influences the body, it is less effective than the sine wave. Additionally, as we will see in the next chart, the nature of the pulse is such that desired impact of the primary frequency is only approximately ⅓ of the effect of the sine wave.

FIG. 9 shows a harmonic analysis of the two approaches. The sinusoidal approach is represented by curve [1], in the frequency domain [4], with its peak at the expected 7.83 Hz, with no harmonics. Curve [2] shows the harmonics of the pulse approach. It is equivalent to a long series of sinewaves of increasing frequency, with the prime frequency component having about ⅓ the magnitude of the sinusoidal approach.

FIG. 9 curve [3] shows the same approach, with the RMS of the pulse increased by a factor of 20 so as to have the same effect proportionally as the sinusoidal approach. A consequence of this is that all the harmonics also increase in magnitude (Gauss). Understanding that each harmonic of increasing frequency becomes more effective at influencing the object or area by nature of the derivative of the harmonic being proportional to the frequency. This is detrimental to the goal of encouraging a wave response in the intended object or area of only the prime frequency.

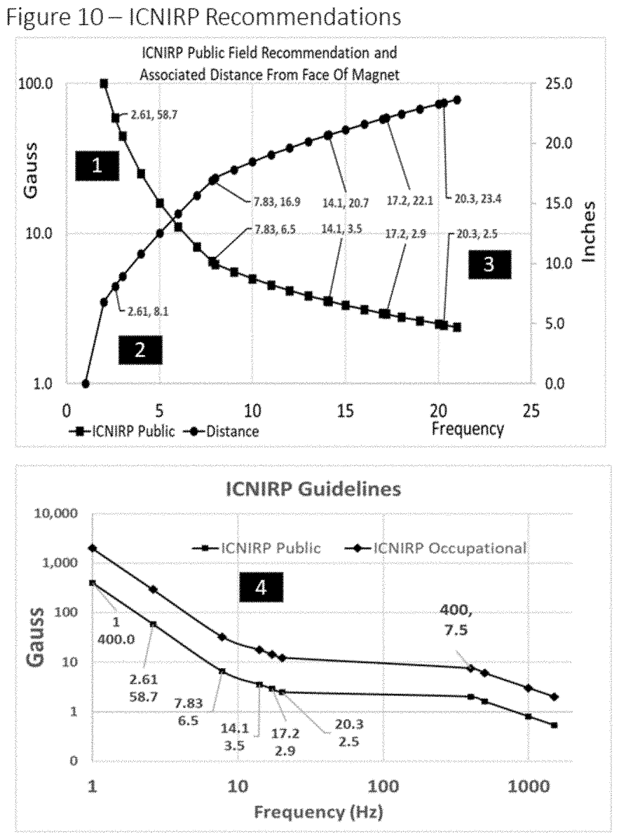

FIG. 10 provides guidance for the basis of the nominal distance of 3 feet for use, with an operating range from 2 feet to 5 feet. 22.1 inches is the greatest distance given the operating parameters of the Sphere limited to 17.2 Hz, making 24 inches a reasonable basis, especially since the guidelines already incorporate significant error margins biased conservatively. 17.2 Hz is the mid-point between the second and third natural resonance of the earth. The curve is developed from the frequency bands and associated equations for guideline limits from ICNIRP—The International Commission On Non-Ionizing Radiation Protection, Published in Health Physics 99(6):818-836; 2010.

### Advantages

Reduced Energy.

Less energy is required by the user to sustain a magnetic field. To create a magnetic field to encompass, for example, a king-sized bed, to fluctuate between 0.2 and 1.0 gauss would require huge coils and large currents, or many turns of wire and high voltage. This invention requires neither.

Safety.

Since the wave is radiated by a moving or spinning object, the wave closely emulates what nature itself produces (which is also a spinning object). Steep step-change electrical pulses in conventional systems' coils may produce harmful effects not present in this invention (as discussed above). The inertia of the magnet used to radiate the field significantly reduces any pulses that may exists within the drive mechanism of the magnet, the drive mechanism being magnetic or other. The object or area experiences a sinusoidal, analog (not digital) wave. Again, not requiring high voltage or current, being powered by a typical, inexpensive USB power source (battery or plug-in), makes the unit safer as well.

Placement.

The small size of the unit allows it to be placed unobtrusively under a bed, on a nightstand, under a couch. The current embodiment has a nominal effective range of 2 to 5 feet, but this is only limited by the strength of the magnet used to radiate the magnetic field. With this range, it can even be placed behind a wall adjacent to the headboard of a bed, and be effective, with nothing in the room itself. This lends itself to other uses as well where an oscillating magnetic field may be desired, without placing something immediately in the environment of interest.

| FIG. # | Title | Reference Numerals |
|---|---|---|
| 1 | Embodiment #1: | 10 - Two 3″ magnets |
| | Proof of Concept | 11 - Axle, non-magnetic |
| | | 12 - Geared transmission |
| | | 13 - DC motor |
| 2 | Embodiment #2: | 20 - Two 3″ magnets |
| | Horizontal Coil | 21 - Drive coil |
| | Approach | 22 - Magnetic Offset Screw |
| | | 23 - Axle |
| | | 24 - Circular housing |
| | | 25 - Electronic module to drive coil. |
| 3 | External Two | 30 - 3″ magnets (2) |
| | Coil Approach | 31 - Drive coils (2) |
| | | 33 - Electronic module to drive coil. |
| | | 34 - Circular Housing |
| 4 | Vertical | 40 - 3″ magnets (2) |
| | Approach | 41 - Axle |
| | | 42 - Nylon journal bearing |
| | | 43 - Drive coils (4) |
| | | 45 - Electronic module to drive coil. |
| | | 44 - Circular Wood Base |
| | | 45 - Electronic module to drive coil. |
| 5 | Magnetic Flux | 1 - Drive coil voltages (flux). |
| | Developed at 9 | 2 - Flux radiating from base of unit. |
| | Inches From Base. | 3 - Volts - drive coil, left axis, |
| | No Magnets | TriField Magnetic sensor right axis. |
| | | 4 - single drive coil flux harmonics |
| | | 5 - combined flux harmonics at 9 |
| | | inches. |
| 6 | Magnetic Flux | 1 - Drive coil voltages. |
| | With Magnets, | 2 - TriField Magnetic sensor at two |
| | at 2 Feet | feet |
| | | 3 - Voltage induced in the |
| | | characterizing coil, a 6″ diameter |
| | | air-core coil. |
| | | 4 - Harmonics of one of two drive |
| | | coils, both having the same |
| | | harmonics, offset by 90 degrees. |
| | | 5 - TriField Magnetic sensor |
| | | harmonics. |
| | | 6 - Characterizing coil harmonics. |
| 7 | Delta-Gauss Per | 1 - Data points collected radially, or |
| | Revolution - Current | axially. |
| | Embodiment | 2 - Trend curves developed to test |
| | | the data. |
| | | 3 - The power function curve and |
| | | the coefficient of determination. |
| 8 | Wave Vs Pulse - | 1 - Gauss curves for equivalent |
| | Wave Analysis | RMS values, pulsed versus sine |
| | | wave |
| | | 2 - Rate of Change of Gauss, that |
| | | being proportional to induced |
| | | effects, |
| 9 | Effect: Wave Vs | 1 - Harmonic analysis of the wave |
| | Pulse | approach. |
| | | 2 - Harmonic analysis of the pulse |
| | | approach when the RMS values of |
| | | the wave and the pulse are |
| | | equivalent. |
| | | 3 - Harmonic analysis of the pulse |
| | | approach when the RMS value of |
| | | the wave is increased to achieve a |
| | | proportionally equivalent effect to |
| | | the wave approach. |
| | | 4 - Frequency domain axis with |
| | | primary peak at 7.83 Hz. |
| 10 | ICNIRP | 1 - Logarithmic axis/curve of |
| | Recommendations | recommended Gauss for a given |
| | [International | frequency of magnetic wave. |
| | Commission On | 2 - Distance from the face of the |
| | Non-Ionizing | magnet for the associated Gauss |
| | Radiation: | 3 - Selected frequency-based pairs |
| | Guidelines For | of Gauss and Distance as employed |
| | Limiting Exposure | in the current embodiment. |
| | to Time-Varying | 4 - Comparison of Occupational and |
| | Electric and | Public guidelines for all frequencies |
| | Magnetic Fields | discussed herein. |
| | (1 HZ to 100 kHz)] | |

The device radiates a varying purely magnetic field of a specific frequency, or a program of specific frequencies, using a novel approach of a magnet or magnet array as the source of the radiated magnetic field. The type and strength of the magnets, the shape of the array and the means by which the magnets are moved may vary depending upon the embodiment as applied to specific uses.

FIG. 1 shows the very first inception of the concept. The motor's current was controlled via regulated power supply to limit the speed of the magnets through a geared transmission, thereby controlling the frequency of the radiated field.

When building this, time constraints drove the inventor to build the Serenity Wave Halo version of Serenity Wave.

FIG. 2 shows the first electronically controlled magnet array. The coil produces a weak magnetic field sufficient to turn the magnet over. A magnetic screw is used to tilt the rotor to ensure the magnet array does not come to rest at a zero-torque point. This was a logical progression as it allowed the inventor to use the hardware from Serenity Wave Halo units to drive the coil Serenity Sphere, this invention.

FIG. 3 employs two coils to ensure starting torque, removing the need for a magnetic offset screw. This required the original hardware for the Halo to be modified to support driving two coils independently but still allowed the inventor to capitalize on existing Serenity Wave hardware.

FIG. 4 shows the rotation shifted to a vertical axle approach which is the present embodiment. A magnetic lock between the coils and the rotating magnet array is achieved allowing the electronic module to precisely control the frequency of the radiated magnetic field. Opposite coils in the base are energized in tandem with polarities designed to attract the magnet array to face them. The coils are counterintuitively wound on non-ferrous materials (wood) allowing them to use less current to maintain movement. Then the other pair are energized, turning the magnets 90 degrees. In some embodiments, the coils are hidden just below the surface, not visible.

FIG. 5 illustrates a typical magnetic control field used to drive the magnet array. Magnets are not installed in the unit for these measurements. The data for this, and several subsequent graphs is empirical, recording actual running units of the invention. The figure shows the weak, harmonic-riddled magnetic field resulting from pulses in a coil. Subsequent figures show these are removed in end use.

FIG. 6 shows the radiated magnetic flux at a distance of 2 feet in relation to the flux developed in the coils. The magnets are installed for this reading. The top graph shows the voltage/flux square wave used by the coil, and the sinusoidal wave form of both the magnetic field, and induced voltage in a sensing coil, at 2 feet. The bottom graph is a harmonic analysis of those waveforms showing the magnet's field is essentially eliminating the harmonics. The magnetic field of the base alone could not be measured with an instrument capable of seeing 20 mG. An astute observer will note the magnetic lock achieved between the magnet (induced EMF resulting from the movement of the magnet) and the signal driving the coils array (the square waves). This is how amplification and radiating of the control signal is achieved—via magnetic lock.

FIG. 7 provides an analysis of empirical data showing the relationship between distance and magnet flux, and the curve trend equation showing the expected behavior validating the measurements. These were measured with an Alpha Labs magnetometer calibrated to be able to see greater than 0.02 Gauss. The readings were taken out from the faces of the magnets (radial), and up from the magnets (axial). The data was then analyzed to see if it fit the expected drop-off given the geometry. The trend curve equations show that with an inverse cube function, the coefficient of determination was >0.999 for both data sets, as expected. This approach and data are used to size the magnets and establish distances for various embodiments.

FIG. 8 is included to help create an understanding of why this approach works and why people who have a unit in their bedroom or office thank the inventor. The concepts in FIGS. 8 and 9 need to be understood to appreciate the advantages of this approach. Curves in chart [1] represent a hypothetical pulsed and a sinusoidal approach to creating a wave with a 0.127 second period in Gauss (left axis). The invention can produce the sinusoidal wave. Their magnitudes were set to have equivalent RMS values. There are only two points on the pulse curve where the slope of the curve is not zero (in Gauss/second, right axis), when the pulse rises, and when it drops back to zero. The sine wave's influence is only zero at the maxima and minima for the wave. The magnetic field's ability to influence an object or area is proportional to the rate of change of the flux (from Faraday's law) and is also proportional to the square of the time the pulse is applied (roots in Newtonian mechanics). The sinewave has a distinct advantage in this regard.

FIG. 9 shows a harmonic analysis of the two approaches. The sinusoidal approach is represented by curve [1], in the frequency domain [4], with its peak at the expected 7.83 Hz, with no harmonics. This is essentially what the invention does at 2 feet or more. Curve [2] shows the harmonics of the pulse approach. It is equivalent to a long series of sinewaves of increasing frequency, with the prime frequency component having about ⅓ the magnitude of the sinusoidal approach. So even though five times as much flux was applied via the pulse, the primary frequency component is only ⅓ the value of the sine wave, making the sine wave more effective. These harmonics are not desirable but are present in many pulsed systems. They are not present in this invention.

FIG. 9, curve [3], shows the same approach, with the RMS of the pulse increased by a factor of 20 so as to have the same effect proportionally as the sinusoidal approach. A consequence of this is that all the harmonics also increase in magnitude (Gauss). Understanding that each harmonic of increasing frequency becomes more effective at influencing the object or area by nature of the derivative of the harmonic being proportional to the frequency. This is detrimental to the goal of encouraging a wave response in the intended object or area of only the prime frequency. If the magnitude (A) of the magnetic field at a distance is given as;

A=Amax*Sin(2*Pi*Freq*t+offset).

The slope, or rate of change of flux, is the derivative;

Amax*2*Pi*Freq*Cos(2*Pi*Freq*t+offset).

The term includes “Freq” as a multiplier. Each of the harmonics detrimental effect increase with the harmonic's frequency. Hence this invention's effort to reduce/eliminate them using a novel approach heretofore not seen. In curve [2], visual inspection of the curve shows you that the 13th peak is 12 times the prime frequency and that its contribution to effects is thus enhanced by a factor of 12 based on frequency alone. They are not present in this invention.

### Embodiments

### First Embodiment—FIG. 1: Proof of Concept

At the conclusion of the inventor's research to help his wife using magnetic therapy, he decided on an approach using powerful magnets placed in motion. However, nobody had thought to use permanent magnets to create oscillating magnetic fields, so he proceeded to build something himself. He purchased powerful neodymium magnets and mechanical items from a local hobby store to build a transmission to couple a small DC motor to the magnets to cause and control rotation. The inventor tried it himself and he had objective results to show the unit helped him during the day. However, after several attempts to build a quieter unit, with constraints of long hours at work, he decided on an electrical magnetic approach versus a mechanical magnetic approach. FIG. 1 shows the first approach of Serenity Sphere, the subject invention of this application.

After designing a system capable of adjusting the magnetic field in a stall, the size of the coil and the voltage required prompted the inventor to develop this invention.

The first “Sphere” was powered from a regulated power supply. The current was adjusted so the frequency radiated by the magnet was the desired 7.83 Hz. This frequency was chosen because it fell with the theta band of sleep, scientific research had shown that exposure to the earth's natural magnetic vibrations were important and improved health and disposition, and we were already being exposed to it.

### Second Embodiment—FIG. 2: Horizontal Coil Approach

Attempting to capitalize on existing Serenity Wave Halo electronic modules, a single coil approach was developed. 4″ PVC pipes and caps purchased at a local hardware store to house the magnet, and the coil was wound on the exterior of the pipe. Various axle and bearing approaches were tried until a quiet reliable solution was arrived at. These are the first units provided to family and customers. Other embodiments existed with aesthetic variations inconsequential to the iteration of the embodiment. They were all single-coil embodiments.

The inventor was able to make a single coil approach work because of his backgrounds in several disciplines, including computer science, electronic and mechanical design. Understanding the physics of rotational inertia and magnetic clock in motors, he was able to write software to control the electronics to take a magnet sitting at rest and without any sensors as to magnet position, was able to achieve magnetic lock and precise frequency control. On the off chance the rotor came to magnetically perfectly perpendicular to the coil, a magnetic off-set screw was applied to tilt the rotor, to ensure starting torque.

However, the unit had to have software designed to check that the magnets were in fact moving because occasionally the rotors would stall, requiring the unit to self-start again. This drove the need for a way to provide more than one axis point of torque.

### Third Embodiment—FIG. 3: External Two-Coil Approach

Using the same PVC pipe, then clear acrylic, two coils were wound as shown. The software was revised to drive two coils and a circuit was added to the electronics to allow for controlling two coils. The need for a magnetic offset screw was eliminated and greater stability of magnetic lock was achieved.

### Fourth Embodiment—FIG. 4: Vertical Approach

In an effort to further refine the silent operation of the axle, a vertical approach was adopted. More advanced plastics were tested along with different types of ball bearings. The current embodiment uses a nylon sleeve as a journal bearing to manage radial loads and a rounded shaft point spinning on a nylon surface to manage axial loads. This proved to be the quietest approach. In delta band and theta band modes, the unit is essentially silent.

The coils in the base are wound on non-ferrous cores and are energized such that the magnet(s) is pulled to alignment between two diametrically opposite coils at all times. Alternate pairs of coils are energized and deenergized as needed to sustain the frequency and motion. The base coils are only strong enough to move the magnet because of their proximity to the magnet. They themselves do not contribute any measurable amount of flux to the user. By being wound on non-ferrous cores, the inertia of the magnet(s) is capitalized upon, eliminating harmonics that would be introduced when passing near a core due to magnetic attraction to the core of an unenergized coil.

Not germane to the technical elements of this embodiment is the fact that this approach allows for many types of decorative or industrial enclosures. Currently a clear acrylic cover is the primary enclosure.

### Future Embodiments

Currently two primary mechanisms have been used to place the magnet(s) in motion and develop constant precise frequencies. One involved an electric hobby motor, the other involved electronics and small drive coils. The next embodiment is currently under development and will be purely mechanical, using energy stored in a spring, and a mechanical governor to control the frequency of the magnets. The unit can be wound up and will run for a sufficient amount of time based on the energy stored in the spring.

### Summary of Embodiments

The patent focuses on using permanent magnets with constant magnetic fluxes to create oscillating magnetic fields, (2) the use of a natural frequency produced by Earth as the basis for providing benefit to users for both sleep and focus, (3) the strength of the oscillating field being of the order of magnitude of the Earth's own field, (4) the simplicity of operation and use, (5) not requiring any electrodes, pads or head bands to be attached to the person or animal using the device, and (6) the application using no obtrusive hardware requirements. The system and methods for magneto-mechanically radiating oscillating magnetic fields for therapy and scientific uses is singularly unique herein.

The device has evolved from simple design and its primary use to aid in sleep, to helping people focus, and to exploring its use to help other mammals, and plants.

Having thus described several embodiments for practicing the inventive method, its advantages and objectives can be easily understood. Variations from the description above may and can be made by one skilled in the art without departing from the scope of the invention.

Accordingly, this invention is not to be limited by the embodiments as described, which are given by way of example only and not by way of limitation.

1. A system for radiating an oscillating magnetic field, comprising:
a magnet array consisting of one or more magnets, the one or more magnetic providing a radiated magnetic field through spinning or other periodic motion of the one or more magnets;
a positioning mechanism for positioning the magnet array within the system; and
a control system used to control the radiated magnetic field,
wherein the system achieves precise control over the radiated magnetic field in a time domain, and in magnitude.

2. The system of claim 1, wherein the control system adjusts the frequency of the radiated magnetic field.

3. The system of claim 1, wherein the magnet array is positioned on a vertical axis within the system via the positioning mechanism, such that the magnets array spins along the vertical axis.

4. The system of claim 1, wherein the magnet array is positioned on a horizontal axis within the system via the positioning mechanism, such that the magnets array spins along the horizontal axis.

5. The system of claim 1, wherein the magnet array is positioned in a periodic, non-rotating manner to change the magnitude of the projected flux, but not its polarity.

6. The system of claim 1, wherein the positioning system includes one, or more coils that when electrically charged cause the magnet array to spin or move periodically.

7. The system of claim 6, wherein the positioning system includes four coils completing two electrical circuits 90 degrees apart, with alternating polarity, such that the coils provide four points of torque per revolution.

8. The system of claim 7, wherein the coils are located beneath the magnet array, producing a weak magnet field powered off a USB power source.

9. The system of claim 1, wherein the system helps a person in need of better sleep and better focus, by stimulating natural waves within the body.

10. The system of claim 1, wherein the system produces a delta-band wave, a theta-band wave and a beta-band wave.

11. The system of claim 1, wherein the control system allows for a repeating motion of the magnetic array and controls the period of the motion of the magnets to provide specific frequencies.

12. The system of claim 1, wherein the magnets are positioned using mechanical methods only, with no use of electrical energy.

13. The system of claim 1, wherein the magnets are positioned using electrical motors or electrical coils.

14. The system of claim 1, wherein the frequency projected by the magnets is held at constant frequency.

15. The system of claim 1, wherein the frequency projected by the magnets is varied to suit scientific or therapeutic objectives.

16. The system of claim 1, wherein the system is used to enhance sleep by replicating natural earth frequencies of the order of magnitude of 7.83 Hz or lower.

17. The system of claim 1, wherein the system is used to enhance mental focus by replicating natural earth frequencies of the magnitude of 17.2 Hz, the midpoint of the second and third natural earth resonance, not to exceed 20.3 Hz, where the third earth resonance is 20.3 Hz.

18. The system of claim 1, wherein the system is used to assist in the therapy of mammals or in the growth of plants.

19. A method of providing a time-varying magnetic field to a subject in need thereof, the method comprising:
providing a magnet array consisting of one or more magnets, a positioning mechanism, and a control system;
using the control system to control the strength of the radiated magnetic field, such that the magnetic array is controlled by the rate of spinning or other periodic motion of the one or more magnets; and
varying the strength of the radiated magnetic field in a time domain.

20. The method of claim 19, wherein the varied strength radiated magnetic field is applied to a user's in order to promote sleep patterns and focus of the user in need thereof.
